(19) United States
(12) Patent Application Publication
(10) Pub. No.: US 2024/0238252 A1
(43) Pub. Date: Jul. 18, 2024

(54) PHARMACEUTICAL COMPOSITION COMPRISING THE COMPOUND SMTP-7

(71) Applicant: Ji Xing Pharmaceuticals Hong Kong Limited, Shanghai (HK)

(72) Inventors: Naoko NISHIMURA, Tokyo (JP); Keiko HASEGAWA, Tokyo (JP); Keiji HASUMI, Tokyo (JP)

(21) Appl. No.: 18/287,489

(22) PCT Filed: Apr. 20, 2022
(86) PCT No.: PCT/US2022/025462

§ 371 Date: Oct. 19, 2023

(30) Foreign Application Priority Data
Apr. 20, 2021 (JP) 2021070892

(51) Int. Cl. A61K 31/407; A61K 9/00; A61K 47/26; A61K 47/44

(52) U.S. Cl. CPC A61K 31/407; A61K 9/0019; A61K 47/26; A61K 47/44

(57) ABSTRACT

This application provides a highly safe pharmaceutical composition containing SMTP-7 or a salt, ester or solvate thereof as an active ingredient, wherein the said pharmaceutical composition further containing either or both of a basic additive and an amphipathic additive. The present invention is useful for prevention or treatment of ischemic disorders such as cerebral infarction including acute ischemic stroke, particularly for treating patients who cannot be treated with thrombolytic drugs.

### CROSS-REFERENCE TO RELATED APPLICATIONS

This application claims priority to Japanese Patent Application Serial No. JP2021-070892, filed Apr. 20, 2021, the disclosure of which is hereby incorporated herein by reference in its entirety for all purposes.

### TECHNICAL FIELD

The present invention relates to a pharmaceutical composition comprising SMTP-7 and a basic additive and/or an amphipathic additive.

### BACKGROUND ART

Ischemic disorder is understood as a pathological condition including all symptoms caused by restricted blood flow in any part of the body. In an ischemic region, cells die due to energy depletion, with the result that the region becomes dysfunctional. As a therapy for ischemia caused by thrombus, a thrombolytic drug and a thrombolytic therapy using the thrombolytic drug have been developed for the purpose of dissolving a thrombus to resupply blood to the ischemic region.

For example, a thrombolytic drug, alteplase (genetically modified tissue-type plasminogen activator, rt-PA) focusing on the action of a plasminogen activator (t-PA), has been developed. Alteplase activates plasminogen into plasmin, which breaks down fibrin serving as a core forming a thrombus (Non-Patent Literature 1).

SMTP (Stachybotrys microspora triprenyl phenol) compounds are a group of compounds having a triprenyl phenol skeleton, produced by a filamentous fungus, and known to have a thrombolysis accelerating effect and an angiogenesis inhibitory effect (Patent Literatures 1 to 3). With respect to the thrombolysis accelerating effect, the following mechanism of action is suggested: an SMTP compound leads to a conformational change of plasminogen, by which the sensitivity of plasminogen to t-PA and the binding of plasminogen to, e.g., thrombi, are enhanced, and then, the dissolution of thrombi is accelerated (Non-Patent Literature 2). SMTP compounds are further reported to have an excellent anti-inflammatory effect (Non-Patent Literature 3), a cell protective effect, and usefulness as a medical agent for ischemic disorders (Patent Literature 4).

### SUMMARY OF THE INVENTION

### Technical Problem

An object of the present invention is to provide a highly safe pharmaceutical composition comprising an SMTP compound, SMTP-7, as an active ingredient.

### Solution to the Problem

The present inventors found that the safety of SMTP-7 is enhanced by using a basic additive and/or an amphipathic additive in combination when SMTP-7 is formulated.

More specifically, the present invention relates to the following [1] to [13].
[1] A pharmaceutical composition comprising a compound represented by formula (I):

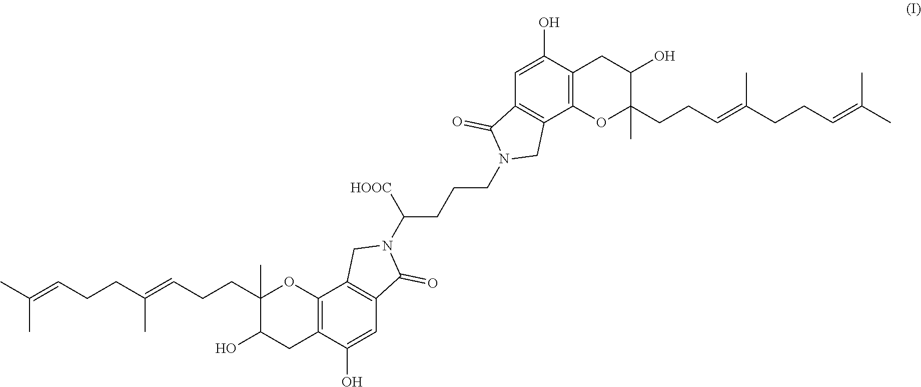 [SMILES: CC(C)=CCC/C(C)=C/CCC1(C)Oc2c(c(O)cc3c2CN(CCCC(C(=O)O)N2Cc4c(cc(O)c5c4OC(C)(CC/C=C(\C)CCC=C(C)C)C(O)C5)C2=O)C3=O)CC1O]

or a salt, ester or solvate thereof, and either or both of a basic additive and an amphipathic additive.
[2] The pharmaceutical composition according to [1], wherein the compound is SMTP-7 represented by formula (II):

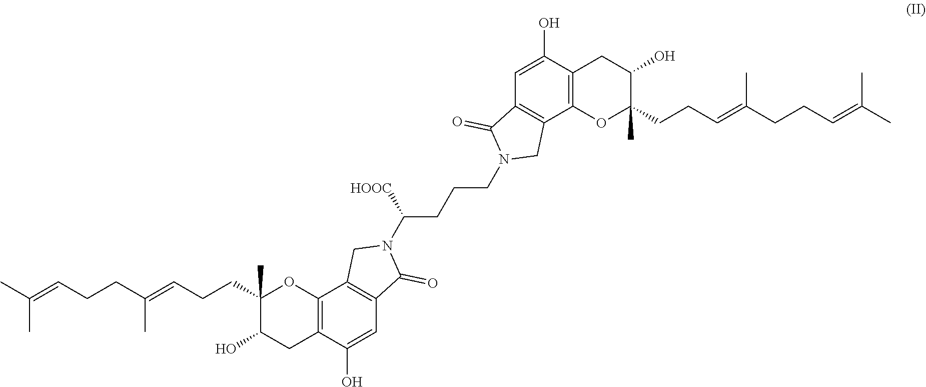 [SMILES: CC(C)=CCC/C(C)=C/CC[C@]1(C)Oc2c(c(O)cc3c2CN(CCC[C@@H](C(=O)O)N2Cc4c(cc(O)c5c4O[C@@](C)(CC/C=C(\C)CCC=C(C)C)[C@@H](O)C5)C2=O)C3=O)C[C@@H]1O]

[3] The pharmaceutical composition according to [1] or [2], which comprises, as the basic additive, one or more selected from the group consisting of amino sugars, alkanolamines and trometamol salts.
[4] The pharmaceutical composition according to [1] or [2], which comprises, as the basic additive, one or more selected from the group consisting of meglumine, triethanolamine and trometamol hydrochloride.
[5] The pharmaceutical composition according to any one of [1] to [4], which comprises, as the amphipathic additive, one or more selected from the group consisting of polyoxyethylene castor oil, polyoxyethylene hydrogenated castor oil, polyoxyethylene sorbitan monolaurate, polyoxyethylene-polyoxypropylene glycol, polysorbate, polyethylene glycol, ursodesoxycholic acid, sorbitan fatty acid ester and sodium desoxycholate.
[6] The pharmaceutical composition according to any one of [1] to [5], which comprises, as the amphipathic additive, one or more selected from the group consisting of polyoxyethylene castor oil, polyoxyethylene hydrogenated castor oil and polyoxyethylene sorbitan monolaurate.
[7] The pharmaceutical composition according to any one of [1] to [6], which comprises both the basic additive and the amphipathic additive.
[8] The pharmaceutical composition according to any one of [1] to [7], which comprises meglumine as the basic additive and polyoxyethylene hydrogenated castor oil as the amphipathic additive.
[9] The pharmaceutical composition according to any one of [1] to [8], which is a formulation for intravenous administration.
[10] The pharmaceutical composition according to any one of [1] to [9], which is used for preventing or treating an ischemic disorder or an inflammatory disease. [11 ] The pharmaceutical composition according to [10], wherein the ischemic disorder is cerebral infarction or transient ischemic attack.
[12] The pharmaceutical composition according to claim [11], wherein cerebral infarction is ischemic stroke.
[13] The pharmaceutical composition according to [12], wherein the ischemic stroke is acute ischemic stroke.
[14] A method for producing the pharmaceutical composition according to any one of [1], [3] to [13], comprising mixing the compound represented by formula (I) or a salt, ester or solvate thereof, with either or both of the basic additive and the amphipathic additive.
[15] A method for producing the pharmaceutical composition according to any one of [2] to [13], comprising mixing the compound represented by formula (II) or a salt, ester or solvate thereof, with either or both of the basic additive and the amphipathic additive.

### Advantageous Effects of the Invention

The present invention provides a safe pharmaceutical composition comprising SMTP-7 as an active ingredient. By virtue of the present invention, SMTP-7 can be applied to a wide variety of targets and used at a wide range of doses, and highly effective medical treatment can be realized.

### BRIEF DESCRIPTION OF DRAWINGS

FIG. 1. The evaluation results of the safety studies in Example 1 the present invention.

FIG. 2. Toxicity scores for pharmaceutical compositions in Example 2 of the present invention.

FIG. 3. Hemolysis levels for pharmaceutical compositions as studied in Example 3 of the present invention.

### DETAILED DESCRIPTION OF THE INVENTION

In the specification, when the numerical range is expressed as A “to” B, the numerical values before and after “to” are included as the lower limit and the upper limit thereof, respectively.

In the specification, when the amount of a component of a composition is described, if a plurality of substances corresponding to the component are present in the composition, the amount refers to a total amount of the substances (present in the composition), unless otherwise specified.

In the specification, the term “step” includes not only an independent step but also an indistinguishable step as long as an intended work can be attained in the step, even if it cannot be clearly distinguished from another step.

In the specification, the “mass %” and “weight %” are interchangeably used, and “parts by mass” and “parts by weight” are interchangeably used.

In the specification, the terms “weight average molecular weight (Mw)” and “number average molecular weight (Mn)” refer to, unless otherwise specified, molecular weights obtained by separating by a gel permeation chromatographic (GPC) apparatus using a column of TSKgel GMHxL, TSKgel G4000HxL or TSKgel G2000HxL (all are trade names, manufactured by Tohso Corporation) and a solvent THF (tetrahydrofuran), and detecting by a differential refractometer, and converted based on polystyrene used as a standard substance.

### 1. Pharmaceutical Composition of the Invention

### 1.1 Components

The pharmaceutical composition according to the present invention is characterized by comprising the compound represented by formula (I), specifically SMTP-7 represented by formula (II), or a salt, ester or solvate thereof, and either or both of a basic additive and an amphipathic additive.

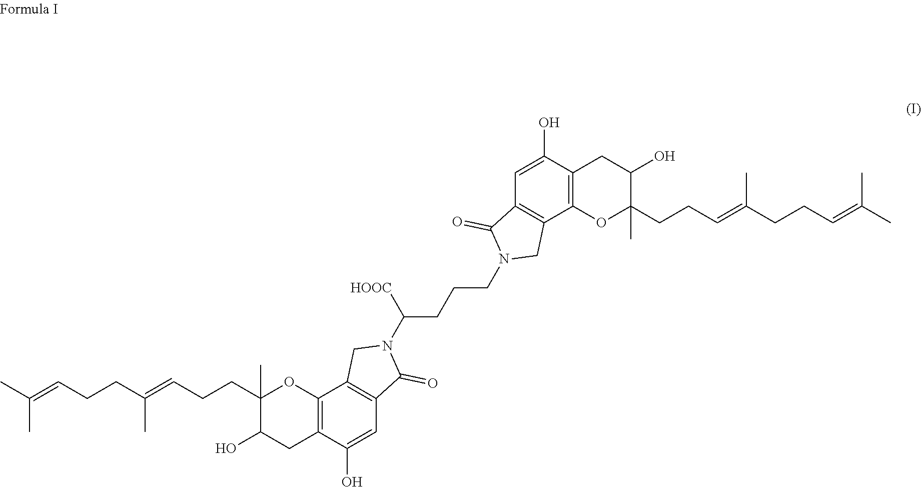 [SMILES: CC(C)=CCC/C(C)=C/CCC1(C)Oc2c(c(O)cc3c2CN(CCCC(C(=O)O)N2Cc4c(cc(O)c5c4OC(C)(CC/C=C(\C)CCC=C(C)C)C(O)C5)C2=O)C3=O)CC1O]

Chemical name: 2,5-bis-[8-(4,8-dimethyl-nona-3,7-dienyl)-5,7-dihydroxy8-methyl-3-oxo-3,6,7,8-tetrahydro-1H-9-oxa-2-azacyclopenta[a]naphthalen-2-yl]-pentanoic acid
Molecular formula: C₅₁H₆₈N₂O₁₀
Molecular weight: 869.09

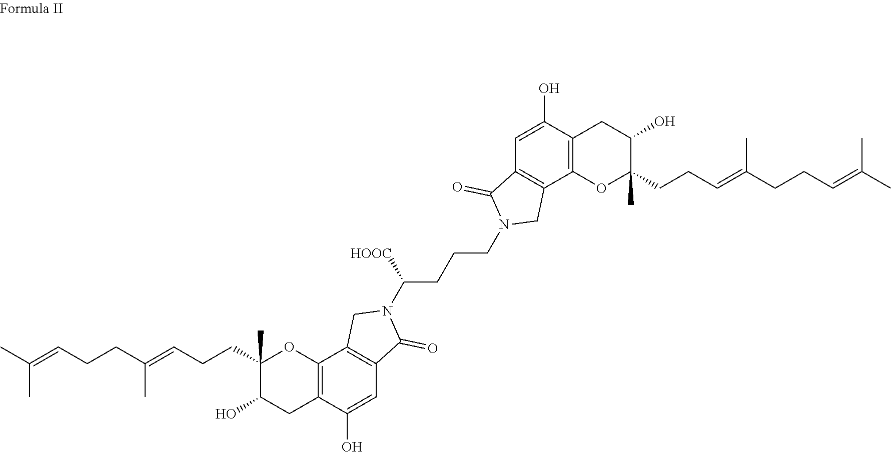 [SMILES: CC(C)=CCC/C(C)=C/CC[C@]1(C)Oc2c(c(O)cc3c2CN(CCC[C@@H](C(=O)O)N2Cc4c(cc(O)c5c4O[C@@](C)(CC/C=C(\C)CCC=C(C)C)[C@@H](O)C5)C2=O)C3=O)C[C@@H]1O]

Chemical name: (S)-2,5-bis((2S,3S)-2-((E)-4,8-dimethylnona-3,7-dien-1-yl) -3,5-dihydroxy-2-methyl-7-oxo-3,4,7,9-tetrahydropyrano[2,3-e]isoindol -8(2H)-yl)pentanoic acid
Molecular formula: C₅₁H₆₈N₂O₁₀
Molecular weight: 868.49

In the specification, unless otherwise specified, the term “SMTP-7” is defined to include not only SMTP-7 but also a salt, ester, or solvate thereof.

### (1) SMTP-7

The compound represented by formula (II) to be used in the pharmaceutical composition of the present invention was developed by the present inventors as a derivative of a compound (SMTP compound) having a triprenyl phenol skeleton and produced by a filamentous fungus. SMTP-7 acts on plasminogen, which is precursor of plasmin present in blood and involved in thrombolysis, to induce a conformational change thereof, and promotes activation by a plasminogen activator to exert a thrombolysis accelerating effect. SMTP-7 is further known to have an anti-inflammatory effect, an antioxidant effect and a cytoprotective effect.

SMTP-7 can be used as a free compound or in the form of a pharmaceutically acceptable salt, ester or solvate. An inorganic acid and an organic acid such as hydrochloric acid, hydrogen bromic acid, sulfuric acid, nitric acid, phosphoric acid or citric acid, and formic acid, fumaric acid, malic acid, acetic acid, succinic acid, tartaric acid, methanesulfonic acid or p-toluenesulfonic acid, are suitable for forming a pharmaceutically acceptable salt of SMTP-7. Also, for example, a compound containing an alkali metal or an alkaline earth metal such as sodium, potassium, calcium or magnesium, a basic amine or a basic amino acid, is suitable for forming a pharmaceutically acceptable salt of SMTP-7. Furthermore, an alcohol or carboxylic acid having 1 to 10 carbon atoms, preferably, e.g., methyl alcohol, ethyl alcohol, acetic acid or propionic acid, is suitable for forming a pharmaceutically acceptable ester of SMTP-7. For example, water is suitable for forming a pharmaceutically acceptable solvate of SMTP-7.

Of the pharmaceutically acceptable salts of SMTP-7 in the pharmaceutical composition of the present invention, esters or solvates (hydrates) mentioned above, a pharmaceutically acceptable salt of SMTP-7 is preferable. More specifically, an alkali metal or alkaline earth metal salt, such as a sodium, potassium, calcium, or magnesium salt of SMTP-7 is preferable, and a sodium salt of SMTP-7 is more preferable.

SMTP-7 may be obtained by chemical synthesis or from a culture of a filamentous fungus such as Stachybotrys microspore, by purification. A method for producing SMTP-7 is described, for example, in Japanese Patent Publication No. 2004-224737, Japanese Patent Publication No. 2004-224738, and International Publication No. WO 2007/111203.

SMTP-7 may be an enantiomer, a diastereomer, a mixture of enantiomers or a mixture of diastereomers, which may be obtained by chemical synthesis or purified from a culture of a filamentous fungus. When SMTP-7 is obtained from a culture of a filamentous fungus by purification, if D-form or L-form amino acid compound is added to a medium for culturing the filamentous fungus, the corresponding isomer can be obtained.

In the pharmaceutical composition of the present invention, the content of SMTP-7 (free compound), although it varies depending on the dosage form of a pharmaceutical composition, is preferably about 1 mass % to 80 mass % and more preferably about 5 mass % to 60 mass % based on the total mass of a pharmaceutical composition. For example, in the case of an injection, which is prepared (reconstituted) when used, the content of SMTP-7 relative to the total mass of the pharmaceutical composition (except the mass of an injection for dilution) is preferably 10 to 80% and more preferably 20 to 60%.

In certain embodiments, the SMTP-7 containing pharmaceutical compositions are liquid formulations. In other embodiments, the SMTP-7 containing pharmaceutical compositions are lyophilized formulations that are lyophilized from a liquid formulation.

In certain embodiments, SMTP-7 in the pharmaceutical composition is at between about 0.5 mg/mL and about 20 mg/mL. In other embodiments, SMTP-7 in the pharmaceutical composition is at between about 1 mg/mL and about 15 mg/mL. In yet other embodiments, SMTP-7 in the pharmaceutical composition is at between about 1 mg/mL and about 10 mg/mL. In some embodiments, SMTP-7 in the pharmaceutical composition is at about 5 mg/mL and about 10 mg/mL. In some embodiments, SMTP-7 in the pharmaceutical composition is at about 10 mg/mL. In yet other embodiments, SMTP-7 in the pharmaceutical composition is at about 0.5 mg/mL, 0.6 mg/mL, 0.7 mg/mL, 0.8 mg/mL, 0.9 mg/mL, 1.0 mg/mL, 1.1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, 10.0 mg/mL, or a higher concentration.

### (2) Basic Additive

Examples of the basic additive to be used in the pharmaceutical composition of the present invention include an alkanolamine such as triethanolamine, monoethanolamine, diisopropanolamine, triisopropanolamine, diethanolamine or 2-amino-2-methyl-1-propanol; trometamol (trishydroxymethylaminomethane (tromethamine)) or a salt thereof; an amino acid such as glycine, arginine, histidine or lysine; an amino sugar such as meglumine or glucosamine; and an amine such as triethylamine, ethylenediamine, epolamine or guanidine.

Of them, one or more selected from the group consisting of an amino sugar, an alkanolamine and a trometamol salt are preferably contained; one or more selected from the group consisting of meglumine, triethanolamine and trometamol hydrochloride are more preferably contained, either one of meglumine and trometamol hydrochloride is more preferably contained; and meglumine is further preferably contained.

When a pharmaceutical composition contains a basic additive, the content of the basic additive, although it varies depending on the dosage form of the pharmaceutical composition, is preferably 0.1 mass % to 50 mass %, and more preferably 1 mass % to 30 mass % based on the total mass of the pharmaceutical composition. For example, in the case of an injection, which is prepared (reconstituted) when used, the content of the basic additive relative to the total mass of the pharmaceutical composition (except the mass of an injection for dilution) is preferably 1 to 50% and more preferably 5 to 30%.

The content of a basic additive relative to the mass of SMPT-7 is preferably 5 mass % to 80 mass %, more preferably 10 mass % to 60 mass %, and further preferably 20 mass % to 50 mass %.

In certain embodiments, the basic additive in the SMTP-containing pharmaceutical composition is at between about 1 mM and about 200 mM. In some embodiments, the basic additive in the SMTP-containing pharmaceutical composition is at between about 2 mM and about 100 mM. In some embodiments, the basic additive in the SMTP-containing pharmaceutical composition is at between about 5 mM and about 50 mM. In some embodiments, the basic additive in the SMTP-containing pharmaceutical composition is at between about 10 mM and about 30 mM. In some embodiments, the basic additive in the SMTP-containing pharmaceutical composition is at about 20 mM. In some embodiments, the basic additive in the SMTP-containing pharmaceutical composition is at about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, or a higher concentration.

In some embodiments, the basic additive is meglumine.

### (3) Amphipathic Additive

The amphipathic additive to be used in the pharmaceutical composition of the present invention is not particularly limited as long as it can be used in a pharmaceutical formulation. Any one of an anionic amphipathic additive, a cationic amphipathic additive, an amphoteric amphipathic additive and a nonionic amphipathic additive can be used. In particular, a nonionic amphipathic additive is preferable.

The nonionic amphipathic additive is preferably one or more selected from the group consisting of polyoxyethylene castor oil, polyoxyethylene hydrogenated castor oil (for example, polyoxyethylene hydrogenated castor oil 50 and 60), fatty acid polyoxyethylene sorbitan (for example, polyoxyethylene sorbitan monoolate (polysorbate 80), polyoxyethylene sorbitan monolaurate (polysorbate 20)), polyoxyethylene-polyoxypropylene glycol, polysorbate, polyethylene glycol, ursodesoxychol acid, sorbitan fatty acid ester, sodium desoxycholate, polyoxyl stearate, tyloxapol, polyoxyethylene polyoxypropylene glycol, glyceryl monooleate, glyceryl dioleate, glyceryl trioleate, polyglyceryl-3-diolate and phosphatidylcholine; more preferably one or more selected from the group consisting of polyoxyethylene castor oil, polyoxyethylene hydrogenated castor oil and polyoxyethylene sorbitan monolaurate; further preferably polyoxyethylene castor oil or polyoxyethylene hydrogenated castor oil; and particularly preferably polyoxyethylene hydrogenated castor oil.

The total number of oxyethylene units contained in polyoxyethylene hydrogenated castor oil, although it is not particularly limited, is preferably 2 to 150, more preferably 10 to 100, further preferably 40 to 70, particularly preferably 50 or 60, and most preferably 60 (polyoxyethylene hydrogenated castor oil 60).

When the pharmaceutical composition contains an amphipathic additive, the content of the amphipathic additive, although it varies depending on the dosage form of a pharmaceutical composition, is preferably 1 mass % to 80 mass %, more preferably 5 mass % to 60 mass %, and further preferably 5 mass % to 50 mass % based on the total mass of the pharmaceutical composition. For example, in the case of an injection, which is prepared (reconstituted) when used, the content of the amphipathic additive relative to the total mass of the pharmaceutical composition (except the mass of an injection for dilution) is preferably 10 to 80%, and more preferably 20 to 60%.

The content of an amphipathic additive relative to the amount of a SMPT compound is preferably 40 mass % to 300 mass %, more preferably 60 mass % to 200 mass %, and further preferably 70 mass % to 150 mass %.

In certain embodiments, the amphipathic additive in the SMTP-containing pharmaceutical composition is at between about 0.001% and about 10% (w/v). In some embodiments, the amphipathic additive in the SMTP-containing pharmaceutical composition is at between about 0.01% and about 10% (w/v). In some embodiments, the amphipathic additive in the SMTP-containing pharmaceutical composition is at between about 0.01 and about 1% (w/v). In some embodiments, the amphipathic additive in the SMTP-containing pharmaceutical composition is at between about 0.01% and about 1% (w/v). In some embodiments, the amphipathic additive in the SMTP-containing pharmaceutical composition is at between about 0.01%, about 0.1%, or about 1% (w/v). In some embodiments, the amphipathic additive in the SMTP-containing pharmaceutical composition is at about 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1.0%, 1.1%, 1.2% , 1.3%, 1.4%, 1.5%, 1.6%, 1.7%, 1.8%, 1.9%, 2.0%, 3.0%, 4.0%, 5.0% (w/v), or a higher concentration.

In some embodiments, the amphipathic additive is polyoxyethylene hydrogenated castor oil.

Although it is not limited, a preferable example of the pharmaceutical composition of the present invention contains SMTP-7 or a salt thereof (preferably sodium salt), meglumine as a basic additive and polyoxyethylene hydrogenated castor oil as an amphipathic additive.

### (4) Other Additives

The pharmaceutical composition of the present invention may contain other pharmacologically acceptable carriers (additives), which are appropriately selected depending on the usage of the pharmaceutical composition (target disease) and the dosage form of the pharmaceutical composition.

When a pharmaceutical composition contains other additives, the content of the other additives, although it varies depending on the dosage form of the pharmaceutical composition, is preferably 0.1 mass % to 80 mass %, and more preferably, 1 mass % to 60 mass % based on the total mass of the pharmaceutical composition.

### 1.2 Dosage Form

The dosage form of the pharmaceutical composition of the present invention, although it is not particularly limited, is preferably a parenteral formulation and preferably an intravenous formulation (injection). When used as an intravenous formulation, the pharmaceutical composition may be a formulation prepared when used or a ready-to-use formulation.

The pharmaceutical composition, when it is used as an intravenous formulation, may contain, an aseptic aqueous or non-aqueous solution in addition to, e.g., SMTP-7, a basic additive and an amphipathic additive as mentioned above. As the aqueous solvent, distilled water for injection or saline may be mentioned. As the non-aqueous solvent, for example an alcohol such as ethanol may be mentioned. Other than this, the pharmaceutical composition of the present invention may further contain a pharmacologically acceptable carrier such as a tonicity agent, a preservative, a wetting agent, an emulsifier, a dispersant, a stabilizer, a pH regulator or a dissolution aid.

### 1.3 Dosage and Administration

SMTP-7 is known to have not only a thrombolysis accelerating effect but also an anti-inflammatory effect and an antioxidant effect, and exert a cell protective effect. Accordingly, the pharmaceutical composition of the present invention has not only a preventive or treatment effect (based on the thrombolysis accelerating effect) on ischemic disorders, but also a preventive or treatment effect (based on the cell protective effect) on ischemic disorders and cell damage (ischemia/recanalization damage) after ischemia/recanalization, as well as a preventive or treatment effect on inflammatory diseases (see, for example, WO2011-004620).

In the specification, “ischemic disorder” refers to a pathological condition including all symptoms caused by restricted blood flow in any part of the body and includes tissue damages (due to ischemia/recanalization) after recovery of blood flow.

The pharmaceutical composition of the present invention is effective for ischemic disorders, in particular, thrombosis. Thrombosis is commonly understood as a clot of blood in a blood vessel. More specifically, examples of thrombosis include transient ischemic attack, disseminated intravascular coagulation, thrombotic microangiopathy, thrombophlebitis, deep vein thrombosis, idiopathic thrombosis, cerebral infarction (cerebral thrombosis, cerebral embolism), myocardial infarction and pulmonary thromboembolism. Of them, cerebral infarction is the disease to which the pharmaceutical composition of the present invention can be suitably applied.

In the specification, cerebral infarction is used interchangeably with ischemic stroke. Ischemic stroke is commonly acute ischemic stroke, which accounts for almost 90% of all strokes.

In the specification, “inflammatory disease” means all pathological conditions caused by inflammation due to an external stimulus or an internal cause. In particular, the pharmaceutical composition of the present invention is preferably used for nephritises such as interstitial nephritis, pyelonephritis and glomerular nephritis; inflammatory bowel diseases such as Crohn's disease and ulcerative colitis; and autoimmune peripheral neuropathies such as Guillain-Barre syndrome and neuropathy in chronic inflammatory degenerative polyradiculoneuritis (CIDP) (see, WO2007/040082, WO2011/125930).

The dose of the pharmaceutical composition of the present invention, although it varies depending on the use (target disease) and dosage form, is preferably 0.01 mg/kg to 100 mg/kg and more preferably, 0.1 mg/kg to 30 mg/kg generally per adult per time in SMTP-7 (free form) equivalent. The frequency of administration is not particularly limited, and single administration, repeated administration or continuous administration may be acceptable. The administration interval and administration period can be selected by those skilled in the art depending on clinical findings, imaging findings, hematological findings, comorbidities and medical history.

The dose of the pharmaceutical composition of the present invention per adult per day, although it varies depending on the type of SMTP-7, severity of an ischemic disorder and affected area of the body, is preferably 0.01 mg/kg to 100 mg/kg, and more preferably 0.1 mg/kg to 50 mg/kg in SMTP-7 (free form) equivalent.

In the case of repeated administration, the administration schedule: immediately after the onset, within 12 hours after onset and then once per day for about 7 days, is preferable. In the case of continuous administration, administration of 1 to 24 hours per day is preferable.

A method for administering a pharmaceutical composition is not particularly limited and can be selected from various administrations, such as intravenous administration, subcutaneous administration, intramuscular administration and oral administration. For example, in an acute-phase disease, in order to administer a desired dose immediately and securely to the patient, intravenous administration, more specifically, intravenous injection or infusion, is preferable. For example, administering 10% of a single dose as a bolus and 90% of the dose by infusion over 30 minutes to one hour can be employed as the administration method.

The pharmaceutical composition of the present invention may be used in combination with another medical agent as long as the object of the invention can be attained. As the other drug, for example, a thrombolytic drug is mentioned. Examples of the thrombolytic drug to be used in combination include alteplase, urokinase, desmoteplase and monteplase.

After the onset of cerebral infarction, some patients are likely to bleed due to damage on a blood vessel (for example, at the time of reperfusion). In contrast, the pharmaceutical composition of the present invention has a cell protective effect. Due to this, the composition effectively prevents transition to such a hemorrhagic state and reduces tendency to bleed.

The pharmaceutical composition of the present invention is effectively applied to patients to which application of a thrombolytic drug is cancelled for the reason of the lapse of time after the onset. For example, if the pharmaceutical composition of the present invention is applied to a patient with cerebral infarction 3 hours or more after onset and who cannot receive a treatment with administration of e.g., alteplase, an ischemic disorder is sometimes suppressed. Note that, when the onset time is not clearly known, the latest symptomless time (the latest time when a patient was confirmed as symptomless) is deemed as the onset time.

When the pharmaceutical composition of the present invention is applied to a thrombosis (including thrombotic embolism), it is possible to apply the composition to a patient for which a thrombolytic drug is contraindicated. Alternatively, the pharmaceutical composition of the present invention can be applied to a patient to which administration of a thrombolytic drug was cancelled for the reason of appearance of contraindicated signs or symptoms during administration of a thrombolytic drug. Examples of the contraindicated signs include hemorrhage diathesis, hemorrhage, hypertension and impaired blood glucose (control). Also, the pharmaceutical composition of the present invention can be applied, for example, to a patient with cerebral infarction who cannot receive a treatment with administration of a thrombolytic drug because the patient has a great risk of intracranial hemorrhage.

When the pharmaceutical composition of the present invention is used for thrombosis (including thrombotic embolism), it is effective to apply the composition to a patient who usually cannot receive a treatment with a thrombolytic drug. Examples of such a patient include patients having, e.g., transient ischemic attack, disseminated intravascular coagulation, thrombotic microangiopathy, thrombophlebitis, deep vein thrombosis and idiopathic thrombosis.

The use of the pharmaceutical composition of the present invention is not limited in humans and the composition may be used for domestic animals such as cows, horses and sheep, and pets such as dogs, cats and monkeys.

### 2. Method for Making the Pharmaceutical Composition

A method for making the pharmaceutical composition of the present invention includes mixing SMTP-7 with a basic additive and/or an amphipathic additive as mentioned above. A method for mixing them is not particularly limited and a method known in the technical field may be used. If the pharmaceutical composition contains another pharmacologically acceptable carrier (additive), the carrier may be mixed together with the basic additive and/or amphipathic additive in the mixing step.

The mixture of SMTP-7 and additives may be lyophilized and put in ampules or vials and sealed airtight. When used, the lyophilized formulation is reconstituted with a small amount of an injection solution, further appropriately diluted with an injection for dilution such as saline and put in use. Alternatively, the pharmaceutical composition of the present invention may be a ready-to-use formulation produced in advance so as to have an appropriate concentration

### 3. Method for Treating or Preventing Ischemic Disorder

The present invention provides a method for treating or preventing an ischemic disorder, including administering the pharmaceutical composition of the present invention to a patient in need. The administration subject, dose, administration interval, administration period and administration method of the pharmaceutical composition are as previously illustrated in the description of the pharmaceutical composition of the present invention.

The pharmaceutical composition of the present invention is reduced in stimuli and enables a treatment with more effective and highly safe SMTP-7.

### EXAMPLES

The present invention will be more specifically described by way of the Examples but the present invention is not limited to these Examples. Note that, unless otherwise specified, “%” represents mass %.

### Example 1: Safety Studies of the Pharmaceutical Compositions on Mice

### Preparation of the Pharmaceutical Compositions

SMTP-7 (sodium salt) was obtained by purification from a culture, which was obtained by culturing Stachybotrys microspora IFO30018 strain in a medium containing L-ornithine as an organic amino compound, in accordance with the method described in Japanese Patent Publication No. 2004-224738. The purified SMTP-7 was dried. To the dry-solid SMTP-7, 0.3 mol/L NaOH and physiological saline (0.9 mass % NaCl) were added to prepare a 50 mg/ml solution. Then, the solution was adjusted to have a concentration of 10 mg/ml and low pH (weak alkali) by using 0.3 mol/L HCl and physiological saline. The solution was sterilized by filtration, divided into small portions and cryopreserved at −30° C.

The SMTP-7 were obtained in different lots and designated as SM0X02, N152403-01, GMP-like and N01YH/G01HN, respectively.

### Basic Additive, Amphipathic Additive, and the Reference Compound

The basic additive, amphipathic additive and reference compounds used herein are as follows:
Meglumine (Cat. No. M9179, manufactured by SIGMA-ALDRICH, St. Louis, MO, USA): simply referred to as Meg.
Polyoxyethylene hydrogenated castor oil 60 (NIKKOL HCO-60, manufactured by Nikko Chemicals Co., Ltd., Tokyo): simply referred to as HCO-60 or HCO in the Examples.
Polyethylene glycol 4000 (Macrogol 4000, manufactured by NOF Corporation, Tokyo): simply referred to as PEG.
D-mannitol (D-mannitol injection defined by the Japanese Pharmacopoeia, manufactured by Terumo Corporation, Tokyo): simply referred to as Man.

### Administration to Mice

Pharmaceutical compositions 1 to 8 and comparative pharmaceutical compositions 1 to 6 were prepared respectively by dissolving SMTP-7 and either or both of a basic additive and amphipathic additive in physiological saline, and by dissolving a reference compound in physiological saline. The final concentration of SMTP-7 in the pharmaceutical compositions and comparative pharmaceutical compositions was controlled to be 10 mg/mL. The lot numbers of SMTP-7, and types and concentrations of the basic additives and amphipathic additives and reference compounds in the pharmaceutical compositions and comparative pharmaceutical compositions are listed in Table 1.

The pharmaceutical compositions and comparative pharmaceutical compositions prepared were administered to ICR mice (6 to 12 weeks old) at a ratio (dose) of 10 mL/kg (100 mg/kg of SMTP-7 (free form)) through the tail vein. In administration, 1/10 of each of the pharmaceutical compositions was administered as a bolus, and then, the remaining 9/10 of the composition was continuously administered for 30 minutes.

TABLE 1
pharmaceutical compositions in safety studies of the
pharmaceutical compositions on mice

| | | Basic additive, amphipathic | | |
|---|---|---|---|---|
| | | additive or reference compound | | |
| | SMTP-7 | | Concentration | |
| | | | (final | |
| | Lot | Type | concentration) | |
| Pharmaceutical | N152403-01 | Meg | 20 | mmol/L |
| composition 1 | | | | |
| Pharmaceutical | N152403-01 | HCO + | 1% | (w/v) |
| composition 2 | | Meg | 20 | mmol/L |
| Pharmaceutical | GMP-like | Meg | 20 | mmol/L |
| composition 3 | | | | |
| Pharmaceutical | N01YH/G01HN | HCO | 0.01% | (w/v) |
| composition 4 | | | | |
| Pharmaceutical | N01YH/G01HN | HCO + | 0.01% | (w/v) |
| composition 5 | | Meg | 20 | mmol/L |
| Pharmaceutical | N01YH/G01HN | HCO | 0.1% | (w/v) |
| composition 6 | | | | |
| Pharmaceutical | N01YH/G01HN | HCO | 1% | (w/v) |
| composition 7 | | | | |
| Pharmaceutical | N01YH/G01HN | HCO + | 1% | (w/v) |
| composition 8 | | Meg | 20 | mmol/L |
| Comparative | SM0X02 | Not contained | | |
| pharmaceutical | | | | |
| composition 1 | | | | |
| Comparative | SM0X02 | Man | 5% | (w/v) |
| pharmaceutical | | | | |
| composition 2 | | | | |
| Comparative | N152403-01 | Not contained | | |
| pharmaceutical | | | | |
| composition 3 | | | | |
| Comparative | GMP-like | Not contained | | |
| pharmaceutical | | | | |
| composition 4 | | | | |
| Comparative | N01YH/G01HN | Not contained | | |
| pharmaceutical | | | | |
| composition 5 | | | | |
| Comparative | N01YH/G01HN | PEG | 1% | (w/v) |
| pharmaceutical | | | | |
| composition 6 | | | | |

In the above table, the expression “% (w/v)” refers to the mass (g) of the amphipathic additive or reference compound contained in a pharmaceutical composition (100 mL). The expression of “HCO +Meg” indicates that HCO-60 (0.01% (w/v) or 1% (w/v)) and meglumine (20 mM) are both contained.

### Evaluation Method

States of the mice during administration and about one hour after administration were observed. The items observed are as follows. The results are shown by scores of 0 to a maximum value represented by an integer (shown in parentheses).

### Items Observed

States of mice during administration: acceleration of the permeability and red flare of the tail vein (1), engorgement of the tail (1), respiratory exacerbation (4)

States of mice about one hour after administration: engorgement of the tail (4), reduction of physical activity (8), respiratory exacerbation (8), awakening from anesthesia (4).

The score obtained in the case where each of the SMTP-7 sodium salts of different lots was administered alone (score obtained when comparative pharmaceutical composition 1, 3, 4 or 5 was administered, more specifically, a total score obtained by adding the scores on all observation items) was regarded as 1 and the scores obtained by administering pharmaceutical compositions and comparative pharmaceutical compositions were represented by relative values.

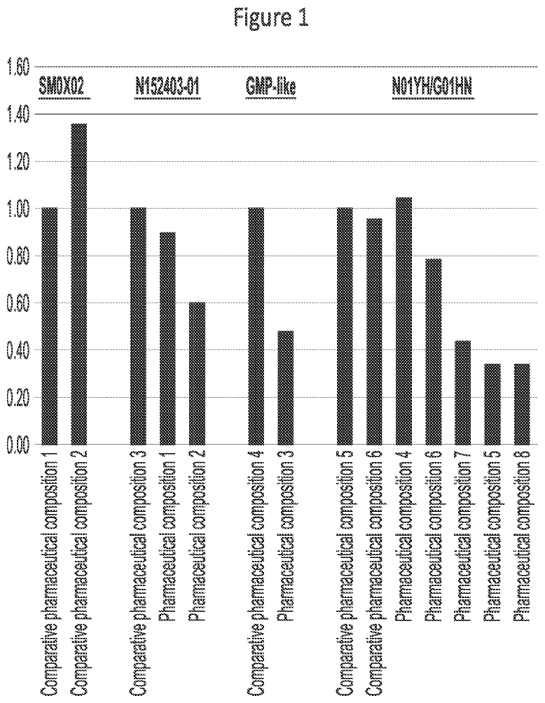

The evaluation results are shown in FIG. 1. In FIG. 1, the longitudinal axis indicates the ratio of the total of scores of observation items in the case where each of the pharmaceutical compositions and comparative pharmaceutical compositions is administered relative to the total of scores of observation items in the case where SMTP-7 sodium salt was administered alone.

From FIG. 1, the followings are found.

When D-mannitol was added to SMTP-7 (lot SM0X02), the score was 1.36 times as high as the reference. In contrast, when meglumine were added to SMTP-7 (lot 152403-01), the score was 0.90 times as high as the reference, and when HCO-60 (1% (w/v)) and meglumine (20 mM, pH8.5) were added in combination, the score was 0.61 times as high as the reference. When meglumine was added to SMTP-7 (lot “GMP-like”), the score was 0.48 times. When HCO-60 (0.01% (w/v)) was added to SMTP-7 (lot “N01YH/G01HN”), the score were 1.05 times. When HCO-60 (0.01% (w/v)) and meglumine (20 mM, pH8.5) were added in combination, the score was 0.34 times. In the cases where HCO-60 (0.1% (w/v)) and HCO-60 (1% (w/v)) were added, the scores were 0.79 times and 0.44 times, respectively. When HCO-60 (1% (w/v)) and meglumine (20 mM, pH8.5) were added in combination, the score were 0.34 times. In contrast, the score in the case where polyethylene glycol 4000 (1% (w/v)) was added to SMTP-7 (lot “N01YH/G01HN”) was 0.95 times as high as the score of the case where polyethylene glycol 4000 was not added.

From the above, it can be understood that SMTP-7 can be more safely used by virtue of the pharmaceutical composition according to disclosure.

### Example 2: Toxicity Studies of the Pharmaceutical Compositions on Rats

SMTP-7 (sodium salt) was prepared as described in Example 1. The SMTP-7 was obtained in different lot and designated as N1533-62-1. The same Meg and HCO-60 were used as in Example 1.

Pharmaceutical compositions 9-11 were prepared respectively by dissolving SMTP-7 and either or both of a basic additive and amphipathic additive in physiological saline. The final concentration of SMTP-7 in the pharmaceutical compositions was controlled to be 10 mg/mL. The lot numbers of SMTP-7, and types and concentrations of the basic additives and amphipathic additives and reference compounds in pharmaceutical compositions 9-11 are listed in Table 2.

TABLE 2
pharmaceutical compositions in toxicity studies
of the pharmaceutical compositions on rats

| | | Basic additive and/or | | |
|---|---|---|---|---|
| | | amphipathic additive | | |
| | SMTP-7 | | Concentration (final | |
| | Lot | Type | concentration) | pH |
| Pharmaceutical | N1533-62-1 | Meg | 20 mmol/L | 9.5 |
| composition 9 | | | | |
| Pharmaceutical | N1533-62-1 | HCO + | 0.01% (w/v) | 9.5 |
| composition 10 | | Meg | 20 mmol/L | |
| Pharmaceutical | N1533-62-1 | HCO + | 1% (w/v) | 8.5 |
| composition 11 | | Meg | 20 mmol/L | |

The pharmaceutical compositions 9-11 were administered to male and female SD rats (6 to 7 weeks old and weighing 162 to 268 g) at a ratio (dose) of 10 mL/kg (100 mg/kg of SMTP-7 (free form)) through the tail vein. In administration, 1/10 of each of the pharmaceutical compositions was administered rapidly, and then, the remaining 9/10 of the composition was continuously administered for 30 minutes.

The drug was administered intravenously into the tail vein via an indwelling needle. Ten percent of the dose volume was administered rapidly (rapid dosing). During rapid dosing, the pharmaceutical compositions were administered using a disposable syringe, indwelling needle, and extension tube. The rapid dosing rate is 1 mL/kg/5 sec. After completion of rapid dosing, the syringe tube was replaced immediately. And the remaining 90% of the dose volume was administered continuously for 30 minutes (sustained dosing). During sustained dosing, the pharmaceutical compositions were administered using an infusion pump (BS-8000, Braintree Scientific Inc.). The sustained dosing rate is 9 mL/kg/30 min.

States of the rats about one hours, about 4 hours, and the next day (approximately 24 hours after drug administration) after administration were observed.

Urinalysis was performed. Urine was collected for 16 hours from administration to the next morning. Fresh urine was collected within 4 hours of autopsy on the day following administration. Autopsy was performed the day after drug administration.

The toxicity was evaluated based on an average toxicity score of 6 experiments for each of pharmaceutical compositions 9-11. The toxicity score is the sum of evaluation items with different weighing factor (in parentheses) as follows.
i. Abnormal injection site (1)
ii. Abnormal urine color (1)
iii. Reddish urine (2)
iv. Injection site edema (1)
v. Injection site dark-purple tail (1)
vi. Injection site red focus or crust (0.5)
vii. Lung dark-red focus (4)
viii. Renal pelvis dilation (2)
ix. Ureter dilation (2)
x. Urinary bladder dark-green content (1)

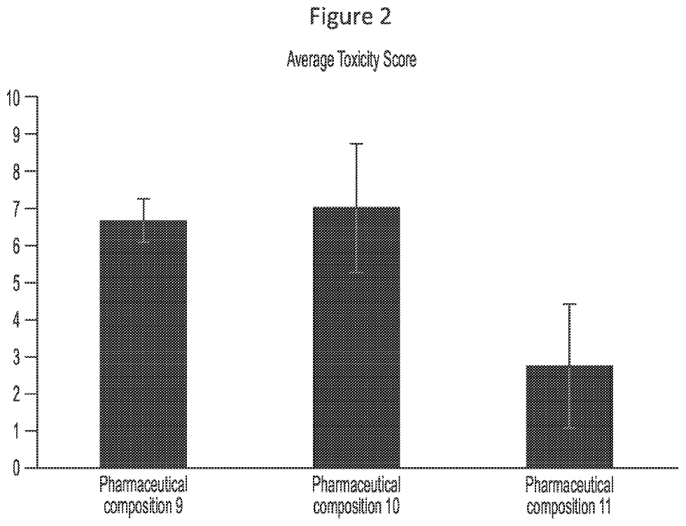

The evaluation results are shown in FIG. 2. In FIG. 2, the longitudinal axis indicates an average toxicity score (the sum of evaluation items) where each of the pharmaceutical compositions 9-11 is administered.

While the toxicity of pharmaceutical composition 9 does not differ from pharmaceutical composition 10. The toxicity of pharmaceutical composition 11 is significantly lower than pharmaceutical compositions 9 and 10.

### Example 3: Hemolysis Studies

Mouse citrated whole blood (100 μl) was mixed with 1 ml of a SMTP-7 containing pharmaceutical composition in saline (200 μM), and the mixture was incubated at 37° C. for 1 min. The SMTP-7 containing pharmaceutical compositions are listed in Table 3 below. SMTP-7 (sodium salt) was prepared as described in Example 1. The same Meg and HCO-60 were used as in Example 1.

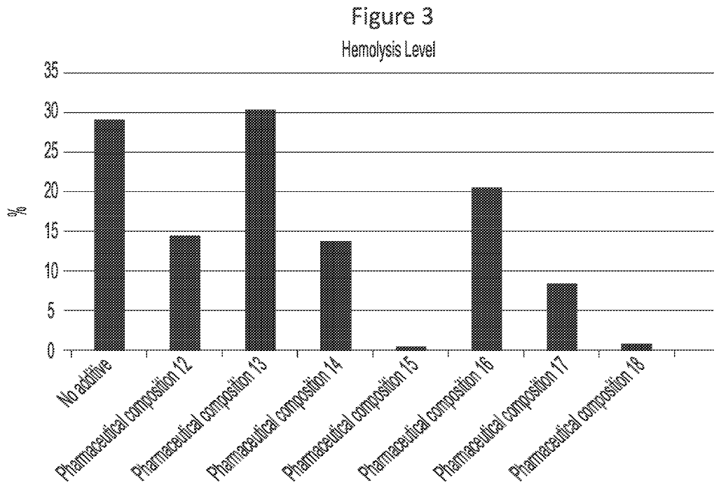

The mixture was then centrifuged at 3,500 rpm for 2 minutes, and the resulting supernatant was diluted 10-fold with saline. Absorbance at 530 nm of the diluted supernatant (100 μl) was measured using a microplate reader. The level of hemolysis (%) was calculated as relative to positive control (100% hemolysis) as shown in FIG. 3.

TABLE 3
SMTP containing pharmaceutical
compositions in the hemolysis studies

| | Basic additive and/or | | |
|---|---|---|---|
| | amphipathic additive | | |
| | | Concentration (final | |
| | Type | concentration) | |
| Pharmaceutical composition 12 | Meg | 20 | mmol/L |
| Pharmaceutical composition 13 | HCO | 0.01% | (w/v) |
| Pharmaceutical composition 14 | HCO | 0.1% | (w/v) |
| Pharmaceutical composition 15 | HCO | 1% | (w/v) |
| Pharmaceutical composition 16 | HCO + | 0.01% | (w/v) |
| | Meg | 20 | mmol/L |
| Pharmaceutical composition 17 | HCO + | 0.1% | (w/v) |
| | Meg | 20 | mmol/L |
| Pharmaceutical composition 18 | HCO + | 1% | (w/v) |
| | Meg | 20 | mmol/L |

### INDUSTRIAL APPLICABILITY

The present invention is useful for prevention or treatment of ischemic disorders such as cerebral infarction including acute ischemic stroke, particularly for treating patients who cannot be treated with thrombolytic drugs.

All publications, patents and patent applications cited in the specification are incorporated herein in their entireties by reference.

### REFERENCES

### Patent Literatures

1. Patent Literatures 1: Japanese Patent Laid-Open No. 2004-224737
2. Patent Literatures 2: Japanese Patent Laid-Open No. 2004-224738
3. Patent Literatures 3: WO2007/111203
4. Patent Literatures 4: WO2011/004620

### Non-Patent Literatures

1. Non-Patent Literature 1: Kano et al., “Hemorrhagic transformation after fibrinolytic therapy with tissue plasminogen activator in a rat thromboembolic model of stroke” Brain Res 2000; 854: 245-248
2. Non-Patent Literature 2: Takayasu et al., “Enhancement of fibrin binding and activation of plasminogen by staplabin through induction of a conformational change in plasminogen” FEBS Letter 1997; 418: 58-62
3. Non-Patent Literature 3: Matsumoto et al., “Soluble Epoxide Hydrolase as an Anti-inflammatory Target of the Thrombolytic Stroke Drug SMTP-7” J Biol Chem 2014; 289: 35826-35838

1. A pharmaceutical composition comprising a compound represented by formula (I): 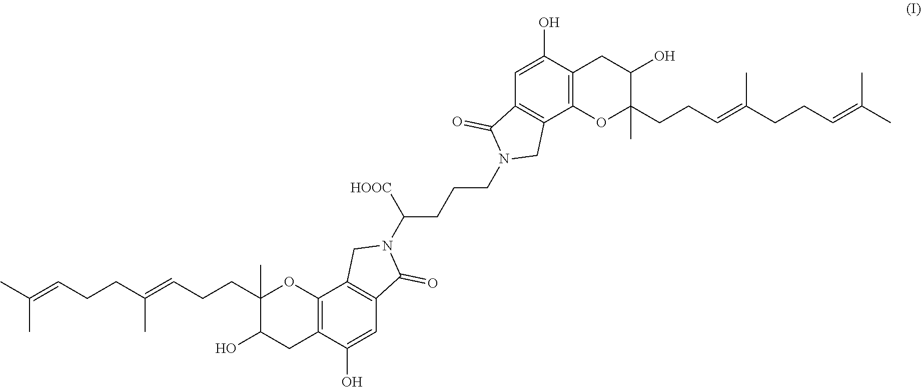 [SMILES: CC(C)=CCC/C(C)=C/CCC1(C)Oc2c(c(O)cc3c2CN(CCCC(C(=O)O)N2Cc4c(cc(O)c5c4OC(C)(CC/C=C(\C)CCC=C(C)C)C(O)C5)C2=O)C3=O)CC1O]
or a salt, ester or solvate thereof, and either or both of a basic additive and an amphipathic additive.

2. The pharmaceutical composition according to claim 1, wherein the compound is SMTP-7 represented by formula (II):

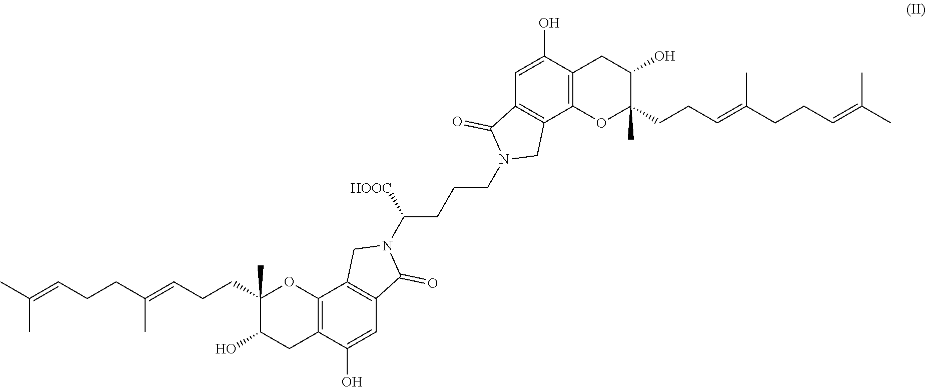 [SMILES: CC(C)=CCC/C(C)=C/CC[C@]1(C)Oc2c(c(O)cc3c2CN(CCC[C@@H](C(=O)O)N2Cc4c(cc(O)c5c4O[C@@](C)(CC/C=C(\C)CCC=C(C)C)[C@@H](O)C5)C2=O)C3=O)C[C@@H]1O]

3. The pharmaceutical composition according to claim 1, which comprises, as the basic additive, one or more selected from the group consisting of amino sugars, alkanolamines and trometamol salts.

4. The pharmaceutical composition according to claim 1, which comprises, as the basic additive, one or more selected from the group consisting of meglumine, triethanolamine and trometamol hydrochloride.

5. The pharmaceutical composition according to claim 1, which comprises, as the amphipathic additive, one or more selected from the group consisting of polyoxyethylene castor oil, polyoxyethylene hydrogenated castor oil, polyoxyethylene sorbitan monolaurate, polyoxyethylene-polyoxypropylene glycol, polysorbate, polyethylene glycol, ursodesoxycholic acid, sorbitan fatty acid ester and sodium desoxycholate.

6. The pharmaceutical composition according to claim 1, which comprises, as the amphipathic additive, one or more selected from the group consisting of polyoxyethylene castor oil, polyoxyethylene hydrogenated castor oil and polyoxyethylene sorbitan monolaurate.

7. The pharmaceutical composition according to claim 1, which comprises, both the basic additive and the amphipathic additive.

8. The pharmaceutical composition according to claim 1, which comprises meglumine as the basic additive and polyoxyethylene hydrogenated castor oil as the amphipathic additive.

9. (canceled)

10. A method for preventing or treating an ischemic disorder or an inflammatory disease in a subject in need of, wherein the method comprises administering a therapeutically effective amount of the pharmaceutical composition according to claim 1 to the subject.

11. The method of claim 10, wherein the ischemic disorder is cerebral infarction or transient ischemic attack.

12. The method of claim 11, wherein cerebral infarction is ischemic stroke.

13. The method of claim 12, wherein the ischemic stroke is acute ischemic stroke.

14. A method for producing the pharmaceutical composition according to claim 1, comprising mixing the compound represented by formula (I) or a salt, ester or solvate thereof, with either or both of the basic additive and the amphipathic additive.

15. (canceled)

16. The method of claim 10, wherein the pharmaceutical is administered intravenously.
